(19) United States
(12) Patent Application Publication
(10) Pub. No.: US 2019/0038128 A1
(43) Pub. Date: Feb. 7, 2019

(54) Remote Based System and Method For Collecting Eye Movement Data

(71) Applicants: Ian Purcell, San Diego, CA (US); Yinhong Qu, San Diego, CA (US)

(72) Inventors: Ian Purcell, San Diego, CA (US); Yinhong Qu, San Diego, CA (US)

(73) Assignee: Dr. Ian Purcell, San Diego, CA (US)

(21) Appl. No.: 15/669,744

(22) Filed: Aug. 4, 2017

(51) Int. Cl. A61B 3/113; G02B 27/01; G06F 19/00; A61B 5/00

(52) U.S. Cl. CPC A61B 3/113; G02B 27/017; G02B 2027/014; A61B 5/0002; G06F 19/3418

(57) ABSTRACT

A remote diagnostic system and method for collecting eye-movement data includes a mobile visual recording device which is mounted on a set of goggles. In this combination, the recording device will necessarily include both a camera and a computer. With the combined visual recording device and goggles stabilized on the head of a patient/user, a blackout vision chamber is established in front of an eye of the patient/user. An illuminator on the goggles can then be activated to illuminate the eye, and the camera in the visual recording device can record eye-movement data from the patient's eye while it is illuminated. A programmed protocol from the computer directs the patient using voice commands through a predetermined sequence of head orientations in space and collects eye-movement data that is used to generate a record of nystagmographs and gyroscopic head positions/stabilization data. The record of eye movements can then be automatically uploaded and viewed remotely by physicians via the web-based browser for diagnostic purposes.

### FIELD OF THE INVENTION

The present invention pertains to systems and methods for collecting eye-movement data. More particularly, the present invention pertains to systems and methods which can be used by a patient/user, on-demand when they are experiencing episodes of vertigo, to collect eye-movement data that can be transmitted to a remote location and presented in a web-based video player simultaneously with nystagmograph datagraphs for clinical evaluation by the patient's healthcare practitioner. The present invention is particularly, but not exclusively, useful for systems and methods which collect eye-movement data using a mobile visual recording device, such as a commercially available smart phone.

### BACKGROUND OF THE INVENTION

Eye-movement data can provide very important diagnostic information for evaluating many different health conditions that may affect a person. For instance, it is known that eye-movement data is diagnostically pertinent for various physiological conditions, such as Benign Paroxysmal Positional Vertigo (BPPV), Migraine, Meniere's Disease, Viral Labyrinthitis, Intracranial Processes (i.e. tumors), and Cardiac Causes. As a practical matter, it typically happens that these conditions are manifested during so-called dizzy spells. Dizzy spells, of course, can happen anywhere and at any time in response to a variety of many different stimuli. Thus, it is desirable for a person who is subject to dizziness to have a diagnostic tool that is readily available, convenient, and easy to use for the purpose of helping identify his/her affliction. This capability, however, requires an ability to visually record eye-movement data during the dizzy spell or, alternatively, in accordance with a predetermined protocol.

These days, a well-known device for taking video pictures is a commercially available visual recording device of a type that is popularly referred to as a smart phone. With this in mind, it has been recognized by the present invention that such a device can be adapted for visually recording episodes of eye movements. Furthermore, it has been recognized that such a device can be configured with an “app” (i.e. computer program) which will format the episodes of eye movements for a proper clinical evaluation and diagnosis.

In light of the above, it is an object of the present invention to provide a system and method for collecting eye-movement data using a mobile visual recording device. Another object of the present invention is to provide systems and methods which can be used for a patient/user, on-demand, to collect eye-movement data for transmission to a remote location where both videos and nystagmographs are simultaneously displayed on a web-based browser for clinical evaluation. Still another object of the present invention is to provide a system and method for collecting eye-movement data which is relatively easy to manufacture, is operationally simple to use, and which is comparatively cost effective.

### SUMMARY OF THE INVENTION

In overview, the present invention requires a headset (i.e. goggles) which is formed for adaptive engagement with a mobile visual recording device (e.g. a smart phone). Once the smart phone has been mounted on the goggles, the combination is then positioned and stabilized on the head of the patient/user. Next, the smart phone is activated to record the patient's eye movements during an episode of vertigo.

As envisioned for the present invention, eye movements can be recorded at any appropriate time, and at any location (e.g. at home, at work, or at a doctor's office). The recorded eye-movement data can then be used to prepare a report that will serve as a diagnostic tool for evaluating the condition of the patient/user. For purposes of the present invention, this report can be prepared using a pre-programmed computer application that is installed in (i.e. downloaded onto) the mobile visual recording device.

Structurally, the present invention includes a base member which is adapted to be held and stabilized on the head of a patient/user. Formed onto the base member is a bracket for holding a visual recording device. Importantly, the visual recording device will need to include both a camera, which defines a camera axis, and a computer. Further, the base member includes an adaptive lens which is used to focus the camera of the visual recording device onto an eye of the patient/user during an operation of the present invention.

With the combined base member and visual recording device positioned and stabilized on the head of a patient/user, a blackout vision chamber is established between the base member and an eye of the patient/user. An illuminator, which is mounted on the base member, is then activated. The purpose here is two-fold. For one, with the patient's eye positioned in the blackout vision chamber, and illuminated by the illuminator, the camera axis of the visual recording device can be aligned with an optical axis of the eye. For another, after an alignment of these axes is established and while the eye is illuminated, eye-movement data can be recorded by the visual recording device.

In detail, for one embodiment of the present invention, the illuminator includes a source of visible light which is incorporated as a component of the visual recording device. It also includes at least one optical fiber for directing visible light from the light source, to the base member and into the blackout vision chamber. For this embodiment of the present invention, the visible light from the light source is directed into the blackout vision chamber via the optical fiber onto a beam path that is oriented at an angle a, transverse to the optical axis of the eye. Preferably, the angle a will be in range between 45° and 90°, to thereby provide for a side-illumination of the patient's eye.

For another embodiment of the present invention, a visible light source may be provided on the base member. In this case, the light source will preferably by a Light Emitting Diode (LED) of a type well known in the art. With this embodiment a switch is provided on the base member for selectively turning the light source on/off. Like the embodiment where visible light is generated by a source on the visual recording device, for this alternate embodiment light will also be directed into the blackout vision chamber on a beam path that is oriented at an angle a transverse to the optical axis of the eye, to thereby provide a side-illumination of the patient's eye.

As indicated above, in an operation of the present invention, the visual recording device records an episode of eye-movement data, and then prepares a report based on this data. For purposes of the present invention, the report will typically include a nystagmogram which records eye movements as a combination of horizontal (x-axis) and vertical (y-axis) measurements. In the nystagmogram, these measurements will correspond to respective deviations of the eye's optical axis from a base reference. Moreover, these deviations are measured simultaneously and they are presented on a same time line as the video of the eye movements. The visual recording device upon completion of the recording session instantaneously uploads the data set including the video eye movements and nystagmographs to a web based server where those data sets are made available for viewing by the patient or patient's physician anytime. As also indicated above, an evaluation of the resultant nystagmogram can provide valuable information regarding a patient's health condition.

In accordance with the present invention, its overall operation is established and controlled by a pre-programmed computer application (i.e. an “app”) which can be downloaded onto the mobile visual recording device. In particular, the computer application will include executable instructions that include visual and voice commands for directing the computer of the visual recording device to perform a process for collecting eye-movement data. These executable verbal and graphic instructions will necessarily include:
verifying a stabilized placement of the base member on the head of a patient to establish a blackout vision chamber in front of an eye, between the base member and the eye of the patient, wherein the mobile visual recording device is mounted on the base member, wherein the camera of the mobile visual recording device defines a camera axis, and wherein the eye of the patient has a visual axis;
activating an illuminator, wherein the illuminator is mounted on the base member for directing visible light into the blackout vision chamber and onto the eye of the patient, and wherein the visible light is directed from the illuminator and into the blackout vision chamber along a beam path that is transverse to an optical axis of the eye, to thereby illuminate the eye during eye movement;
determining when the camera axis of the visual recording device is aligned with the optical axis of the eye, via an adaptive lens mounted on the base member, to focus the visual recording device onto the eye of the patient;
verbally instructing the patient through a predetermined sequence of head orientations in space;
initiating a “GO” signal when each proper head orientation is properly established to receive illuminated eye movement data for a predetermined period of time in each head position;
verbally instructing the patient to hold their head still and keep their eyes open in each of the predetermined head positions
recording illuminated eye-movement and gyroscopic head position data from the patient; and preparing a report based on the recorded eye-movement data that is displayed when both videos and nystagmographs are simultaneously displayed on a web-based browser for remote clinical evaluation

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an exploded perspective view of system components for the present invention, with the components shown in their operative relationship with the head of a patient/user;

FIG. 2 is a view of the present invention as seen in FIG. 1 with the system components combined and operatively stabilized on the head of a patient/user;

FIG. 3A is a schematic presentation of the functional aspects for an embodiment of the present invention using an illuminator mounted in a visual recording device;

FIG. 3B is a schematic presentation of the functional aspects for an alternate embodiment of the present invention using an illuminator mounted on the base member of the system;

FIG. 4 is a cross-section view of the system of the present invention as seen along the line 4-4 in FIG. 2; and

FIG. 5 is a cross-section view of the system of the present invention as seen along the line 5-5 in FIG. 2.

### DESCRIPTION OF THE PREFERRED EMBODIMENTS

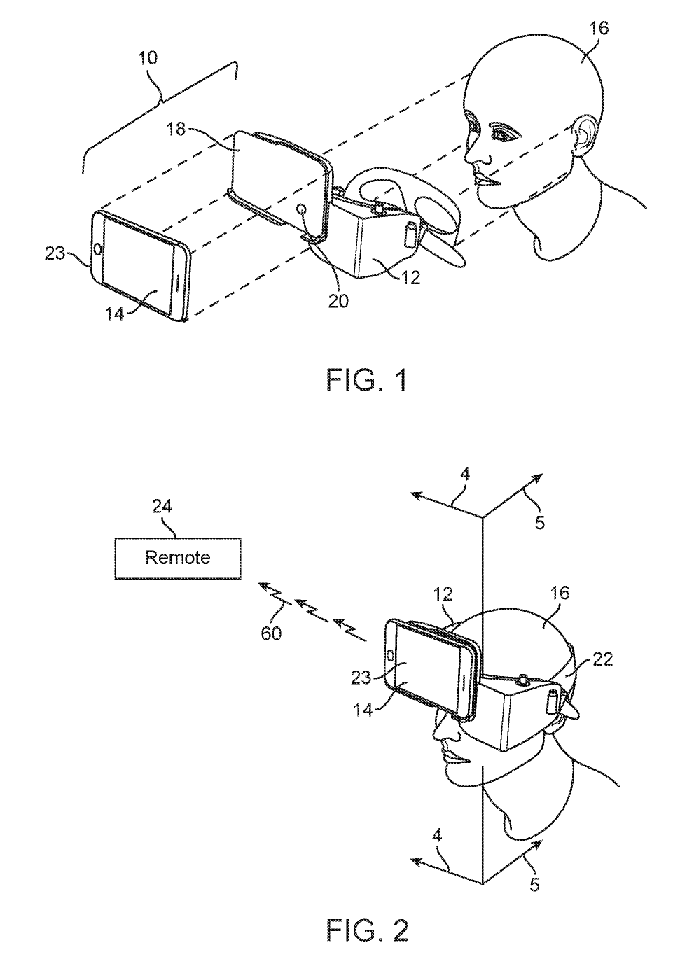

Referring initially to FIG. 1, a system in accordance with the present invention is shown in an exploded perspective view and is generally designated 10. As shown, the system 10 includes a base member 12 and a visual recording device 14 (hereinafter sometimes referred to simply as “device 14”). With reference to both FIG. 1 and FIG. 2, it will be appreciated that the system 10 is intended to be positioned on the head of a patient/user 16 (hereinafter sometimes referred to simply as “patient 16”). Further, in FIG. 1, it will be seen that the base member 12 is formed with a bracket 18, and that it includes an adaptive lens 20. In combination, the bracket 18 is provided to hold the device 14 on the base member 12 and to thereby have the combination function as a pair of goggles. As shown in FIG. 2, the combination of device 14 and base member 12 (i.e. goggles) is to be held and stabilized on the head of the patient 16 by a strap 22. Both FIG. 1 and FIG. 2 show that a gyroscope 23 is also mounted on base member 12 to provide information for determining the three-dimensional, spatial, orientation of the head of patient 16 at any point in time.

With reference to FIG. 2, it is envisioned that the device 14 of system 10 for the present invention can be operationally connected in wireless communication with a remote 24. For purposes of the present invention, the remote 24 may be a database, a clinical location, a medical facility, a person, or some other entity (all not shown) with which/whom the patient 16 desires to communicate. Additionally, or alternatively, along with transmissions to the remote 24, the device 14 can maintain an internal file for any informational data that is obtained during an operation of the system 10.

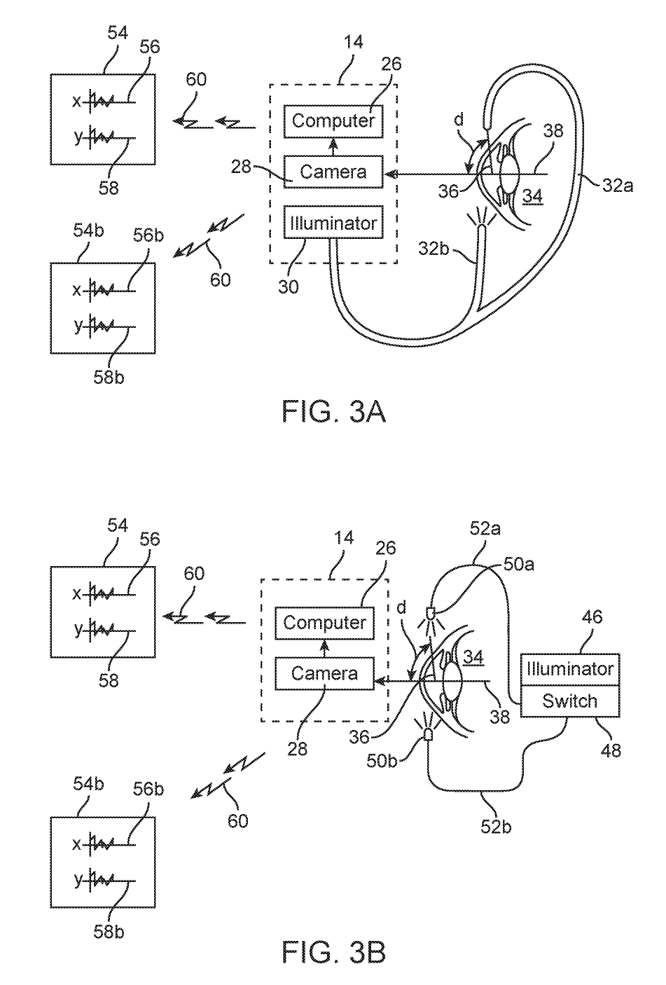

With reference now to FIG. 3A it will be appreciated that the device 14 essentially includes a computer 26 and a camera 28. Still referring to FIG. 3A, it is also to be appreciated that the visual recording device 14 will incorporate an illuminator 30 which includes a source of visible light. For a preferred embodiment of the present invention, visible light from the illuminator 30 is to be transmitted over a pair of optical fibers 32a and 32b. In this combination, visible light from the illuminator 30 is directed via the optical fibers 32a and 32b toward an eye 34 of the patient 16. In detail, this transmission is accomplished to direct visible light along a beam path 36 that is oriented at an angle a relative to an optical axis 38 of the eye 34. For the present invention, the angle a is preferably in a range from 45° to 90°. The eye 34 is thus provided with side-illumination.

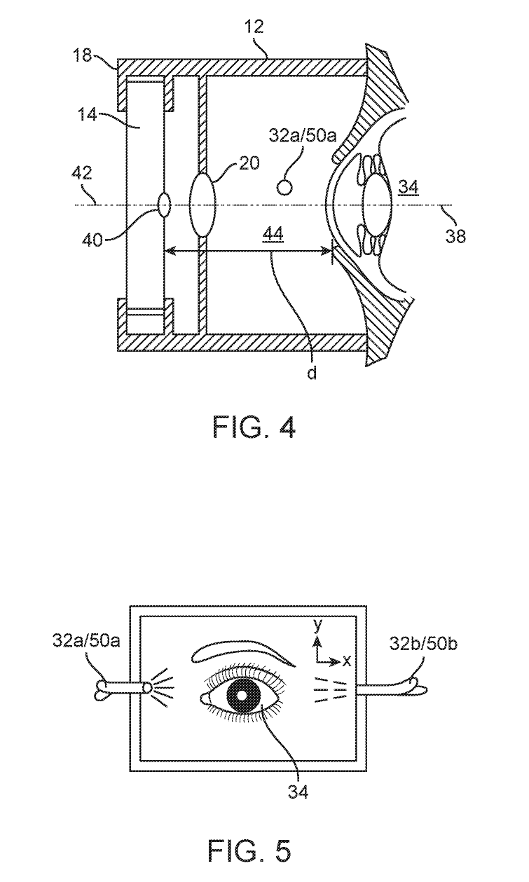

FIG. 4 shows the device 14 in combination with the base member 12 when they are operationally positioned in front of an eye 34 on the head of the patient 16. Also, it will be seen in FIG. 4 that the camera 28 of device 14 includes a camera lens 40 which defines a camera axis 42. Further, FIG. 4 shows that the adaptive lens 20 of base member 12 is positioned between the camera lens 40 and the eye 34 of the patient 16. Importantly, when the combination of base member 12 and device 14 (i.e. goggles) is positioned on the head of the patient 16, two functional requirements are achieved. For one, a blackout vision chamber 44 is created in front of the eye 34. For another, the depth of field for camera 28 of the device 14 is shortened to a distance d. Specifically, this is done to accommodate for the reduced distance between the camera lens 40 and the eye 34. Preferably, the distance d will be in a range between 1.5 cm and 3 cm.

An alternate embodiment of the system 10 for the present invention is shown in FIG. 3B. For this alternate embodiment it is to be appreciated that an illuminator 46 can be mounted on the base member 12, rather than on the visual recording device 14. In this combination for the alternate embodiment, the illuminator 46 is activated by an on/off switch 48 which is connected to two different light sources 50a and 50b via respective connectors 52a and 52b. For purposes of the present invention the light sources 50a and 50b are preferably Light Emitting Diodes (LEDs) of a type well known in the art. Similar to the embodiment of system 10 shown in FIG. 3A, FIG. 3B shows that the light sources 50a and 50b direct visible light along the beam path 36 which is oriented at the angle a relative to the optical axis 38 of the eye 34.

Prior to an operation of the system 10, it is first necessary to establish and verify a properly stabilized position for the device 14 on the head of the patient 16. As intended for the present invention, this will occur when the optical axis 38 of an eye 34 is aligned, and substantially collinear, with the camera axis 42 of camera 28. This can all be done while monitoring camera 28 after the blackout vision chamber 44 has been established, and after the illuminator 30/46 has then been activated. As intended for the present invention, an operation of the system 10 will generate a report that will serve as a diagnostic tool for clinical personnel. In accordance with the present invention, this is accomplished while the eye 34 is side-illuminated as shown in FIG. 5.

In further detail, an operation of the present invention provides the patient 16 with the ability for self-operation, without assistance. To do this, the combination of base member 12 (i.e. goggles) and the visual recording device 14 is positioned on the head of the patient as described above. The visual recording device 14 is then activated, and the illuminator 30/46 is turned on. Visual recording device 14 will then verbally instruct the patient 16 to move his/her head through a predetermined sequence of head positions (orientations).

These positions are:
Body supine with head turned to the right 30°;
Body sitting with head neutral;
Body supine with head turned to the left 30°; and
Body sitting with head neutral.

The Visual Recording Device 14 may also verbally instruct the patient 16 to move his/her head through another predetermined sequence of head positions (orientations).

These positions are:
Body supine and head elevated 30° off earth horizontal axis and head center;
Body supine and head elevated 30° off earth horizontal axis and head yaw 30° to the right;
Body supine and head elevated 30° off earth horizontal axis and head yaw 30° to the left.

In accordance with an operation of the present invention, each head position (orientation) in the predetermined sequence is verified by signals from the gyroscope 23. In particular, this verification the predetermined sequence is verified by signals from the gyroscope 23. In particular, this verification includes signals that indicate: whether the head of the patient 16 is properly positioned (oriented); and whether the proper head position (orientation) has been stabilized for the ten to fifteen second duration required to record eye movements. When verification is completed, the patient 16 will be verbally instructed to move his/her body and head to the next sequential head position (orientation). Once the sequence of eye movement data has been recorded by the visual recording device 14, the data can be transmitted via a wireless link 60 to the remote 24 where it can be viewed by the patient or patient's physician at a later date.

During an eye movement episode at each head orientation, both x and y measurements are simultaneously taken. As shown in FIGS. 3A and 3B, the result of these measurements is a nystagmogram 54 which presents x-measurements 56 on a same time reference with the y-measurements 58 of eye 34. Also during an eye movement episode at each head orientation, both x and y measurements representing head stability are simultaneously collected. As shown in FIGS. 3A and 3B the result of these measurements is a graph 54b which presents x measurements 56b on a same time reference with the y measurements 58b of the patient/user head 16. While the particular Remote-Based System and Method for Collecting Eye-Movement and Head Stability Data as herein shown and disclosed in detail is fully capable of obtaining the objects and providing the advantage herein before stated, it is to be understood that it is merely illustrative of the presently preferred embodiments of the invention and that no limitations are intended to the details of construction or design herein shown other than as described in the appended claims.

### Glossary—File #11665.1

10 system
12 base member
14 visual recording device (eg. Smartphone)
16 patient/user
18 bracket
20 adaptive lens
22 strap
23 gyroscope
24 remote
26 computer
28 camera
30 illuminator
32 optical fibers (a, b)
34 eye
36 beam path
38 optical axis
40 camera lens
42 camera axis
44 blackout vision chamber
46 illuminator
48 on/off switch
50 light sources (a, b)
52 connectors (a, b)
54 nystagmogram
54b graphical head stability data
56 x-measurements
56b x-measurements head stability
58 y-measurements
58b y-measurements head stability
60 wireless or internet connection
62
64
66
68
70
72
74
76
78
80
82
84

What is claimed is:

1. A system for collecting eye-movement data using a mobile visual recording device, wherein the mobile visual recording device includes a camera defining a camera axis, the system compromising:
a base member adapted to be held and stabilized on the head of a patient to establish a blackout vision chamber between the base member and an eye of the patient;
a bracket formed on the base member for holding the visual recording device on the base member;
an adaptive lens mounted on the base membrane for focusing the visual recording device onto the eye of the patient;
an illuminator for directing visible light into the blackout vision chamber and onto the eye of the patient along a beam path transverse to an optical axis of the eye, to illuminate the eye during eye movement; and
a pre-programmed computer application installed in the mobile visual recording device for verbally and graphically directing the patient through a predetermined sequence of head orientations, for receiving illuminated eye-movement and gyroscopic position and head stability data from the patient, and for processing the eye movement and head position/stability data to prepare a report based on the eye-movement and head position/stability data.

2. The system of claim 1 further compromising a gyroscope mounted on the base member for providing head orientation and head stability data to the computer application during the predetermined sequence of head orientations.

3. The system of claim 2 further compromising an audio means mounted on the mobile visual recording device, wherein the audio means is responsive to head orientation data from the computer application for verbally instructing the patient into a correct head orientation during the predetermined sequence of head orientations, and for verifying a proper assumption of the correct head orientation.

4. The system of claim 1 wherein the illuminator comprises:
a light source mounted on the base member; and
an on/off switch mounted on the base member for activating the light source.

5. The system of claim 1 wherein the illuminator comprises:
a light source mounted on the mobile visual recording device for generating visible light; and
at least one optical fiber interconnecting the light source with the blackout vision chamber to provide a side-illumination of the eye.

6. The system of claim 1 wherein the camera axis of the mobile visual recording device is aligned with the optical axis of the eye via the adaptive lens when the mobile visual recording device is engaged with the base member and the base member is held on the head of the patient, wherein the adaptive lens establishes a depth of field d for the mobile visual recording device, and wherein d is in range between 1.5 cm and 3 cm (1.5 cm<d<3 cm).

7. The system of claim 5 wherein an angle a between the beam path and the optical axis of the eye is in a range between 45° and 90° (45°<a<90°).

8. The system of claim 7 wherein the report includes a nystagmogram and a graphical representation of head position/stability data.

9. The system of claim 7 wherein the system is remote-based and the report is transmitted from the visual recording device to a clinical specialist via a web-based browser and video player.

10. The system of claim 1 wherein the mobile recording device is a smart phone and the base member is a pair of goggles with a head strap.

11. A method for collecting eye movement and head position/stability data in real time, using a mobile visual recording device, the system compromising the steps of:
providing a base member, wherein the base member includes a bracket formed thereon for holding a visual recording device, and wherein the bracket is formed with an adaptive lens;
Positioning the visual recording device on the bracket of the base member, wherein the visual recording device includes a camera defining a camera axis;
stabilizing the base member on the head of a patient to establish a blackout vision chamber between the base member and an eye of the patient;
aligning the camera axis of the visual recording device with an optical axis of the eye, via the adaptive lens mounted on the base member, to focus the visual recording device onto the eye of the patient;
activating an illuminator to direct visible light into the blackout vision chamber and onto the eye of the patient along a beam path transverse to an optical axis of the eye, to illuminate the eye during eye movement; and
using a pre-programmed computer application installed in the mobile visual recording device for verbally directing the patient through a predetermined sequence of head orientations, for receiving illuminated eye-movement and head position/stability data from the patient at the head orientation, and for processing the eye-movement and head position/stability data to prepare a report, wherein the report includes a nystagmogram and a graphical representation of head position/stability data.

12. The method recited in claim 11 wherein the predetermined sequence of movements into head orientation includes assuming a position with:
Body supine with head turned to the right 30°;
Body sitting with head neutral;
Body supine with head turned to the left 30°; and
Body sitting with head neutral;
or
Body supine and head elevated 30° off earth horizontal axis and head center;
Body supine and head elevated 30° off earth horizontal axis and head yaw 30° to the right;
Body supine and head elevated 30° off earth horizontal axis and head yaw 30° to the left and further wherein the head is held stationary in each head orientation for ten to fifteen seconds.

13. The method recited in claim 11 wherein the adaptive lens establishes a depth of field d for the mobile visual recording device, and wherein d is in a range between 1.5 cm and 3 cm (1.5 cm<d<3 cm).

14. The method recited in claim 11 further comprising the steps of:
generating visible light; and
directing the visible light into the blackout vision chamber to provide a side-illumination of the eye, and wherein an angle a between the beam path and the optical axis of the eye is in range between 45° and 90° (45°<a<90°).

15. The method recited in claim 11 wherein the method is useful for recording eye-movement data pertinent to an eye condition selected from the group of diagnostic options consisting of Benign Paroxysmal Positional vertigo (BPPV), Migraine, Meniere's Disease, Viral Labyrinthitis, Intracranial Processes (i.e. tumors), and Cardiac Causes, and further wherein the report includes a nystagmogram and head position/stability data, wherein the report is transmitted from the visual recording device to a clinical specialist, at a remote location, for at least one diagnostic option listed for selection from the group by the patient/user's physician, and further wherein the mobile recording device is a smart phone.

16. A non-transitory, computer-readable medium installed onto a computer in a mobile visual recording device, wherein the device includes a camera connected to the computer, and wherein the medium is a pre-programmed computer application having executable instructions stored thereon that direct the computer to perform a process for collecting eye-movement and head position/stability data, the medium comprising instructions for:
verifying a stabilized placement of a base member on the head of a patient to establish a blackout vision chamber between the base member and an eye of the patient, wherein the mobile visual recording device is mounted on the base member, wherein the camera of the mobile visual recording device defines a camera axis, and wherein the eye of the patient has a visual axis;
activating an illuminator to direct visible light into the blackout vision chamber and onto the eye of the patient, and wherein the visible light is directed from the illuminator and into the blackout vision chamber along a beam path transverse to an optical axis of the eye, to illuminate the eye during eye movement;
determining when the camera axis of the visual recording device is aligned with the optical axis of the eye, via an adaptive lens mounted on the base member, to focus the visual recording device onto the eye of the patient;
directing the patient through a predetermined sequence of head orientations;
initiating an audible verbal “GO” signal for each head orientation, when the head orientation has been properly established;
receiving illuminated eye movement and head position/stability data from the patient at each head orientation; and
processing the eye-movement and head position/stability data during the receiving step to prepare a report based on the received eye movement and head position/stability data, wherein the report includes a nystagmogram and graphical representation of the patients' head position/stability data.

17. The medium of claim 16 further comprising an instruction for transmitting the report from the visual recording device to a clinical specialist, at a remote location, and wherein the visual recording device is a smart phone.

18. The medium of claim 16 wherein the adaptive lens establishes a depth of field d for the mobile visual recording device, and wherein d is in a range between 1.5 cm and 3 cm (1.5 cm<d<3 cm).

19. The medium of claim 16 wherein the illuminator comprises:
a light source mounted on the mobile visual recording device for generating visible light; and
at least one optical fiber interconnecting the light source with the blackout vision chamber to provide a side-illumination of the eye, and wherein an angle a between the beam path and the optical axis of the eye is in a range between 45° and 90° (45°<a 90°).

20. The medium of claim 16 wherein the medium is useful for recording eye-movement data pertinent to an eye condition selected form the group of diagnostic options consisting of Benign Paroxysmal Positional Vertigo (BPPV), Migraine, Meniere's Disease, Viral Labyrinthitis, Intracranial Processes (i.e. tumors), and Cardiac Causes.
